(19) United States
(12) Patent Application Publication
(10) Pub. No.: US 2005/0282294 A1
(43) Pub. Date: Dec. 22, 2005

(54) Affinity supports with immobilised protein A

(76) Inventor: Lothar Britsch, Reute (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/928,731

(22) Filed: Aug. 30, 2004

(30) Foreign Application Priority Data
Jun. 17, 2004 (DE) 10 2004 029 341.4

(51) Int. Cl. G01N 33/558; G01N 33/543

(52) U.S. Cl. 436/514

(57) ABSTRACT

The present invention relates to affinity supports with immobilised, covalently bonded protein A, which have a particularly high functional efficiency, wherein protein A is bonded to the support via an aminocarboxylic acid as spacer, to the preparation thereof, and to the use thereof in the purification and analysis of antibodies by affinity chromatography.

The present invention relates to affinity supports with immobilised, covalently bonded protein A, which have a particularly high functional efficiency, to the preparation thereof and to the use thereof in the purification and analysis of antibodies by affinity chromatography.

The use of a wide range of support materials with chemically covalently bonded protein A for the analytical determination and the preparative purification of antibodies from blood plasma or cell cultures has achieved constantly increasing importance in recent decades. This relates both to applications of the said affinity media in medical diagnostics and biomedical research and to the preparation of novel medicaments based on antibodies.

For the said purposes, in particular the preparation of affinity media with immobilised protein A, use has recently been made not only of the natural form of antibody-binding protein A which occurs in the cell wall of Staphylococcus aureus and is known as a pathogeneity factor of these microorganisms, in purified form, but also increasingly also genetically modified, so-called recombinant forms of this protein, which are obtained from cell cultures with correspondingly transformed host cells. Recombinant forms of this type include, for example, the removal of part of carboxyl-terminated amino-acid sequences whose biological function is the binding of protein A to the bacterial cell wall and which is not required for use of the protein as affinity ligand. Recombinant forms of protein A are distinguished, inter alia, by, for example, increased stability to proteolytic degradation or by improved resistance to alkaline solutions.

A common feature of all affinity media with immobilised protein A that have been disclosed to date is that a certain amount of the purified protein is applied to a given surface of porous or nonporous particles or other mouldings by chemically covalent immobilisation. A disadvantageous consequence of this type of immobilisation is that only some of the affine binding sites present in protein A are actually available for temporary binding of the antibody molecules. The maximum amount of antibodies which can theoretically be bound to immobilised protein A is given as follows:

Each protein A molecule has five binding sites for antibodies, localised in the corresponding domains A to E, i.e. the stoichiometric ratio for the binding of IgG to protein A, also known as the efficiency or degree of utilisation of immobilised protein A, is theoretically up to 5.0:1. Taking into account the known relative molecular weights of protein A of about 45,000 and of immunoglobulin G of about 150,000, the weight ratio in the case of occupancy of all affine binding sites of protein A by immunoglobulin G is given by
5×rel. molar mass of IgG/rel. molar mass of protein A=16.7:1

This means that a maximum of 16.7 mg of non-aggregated IgG dissolved in a molecularly disperse manner can be bound affinely to 1 mg of protein A. This has to take place under conditions under which binding of IgG to protein A based on purely ionic interaction between-them does not represent a significant contribution to the total amount bound.

With the support materials with immobilised protein A that have been disclosed hitherto, efficiencies for affine bonding of IgG to protein A of from only 0.5 to 1.5:1 can be achieved. These are thus well below the theoretically possible efficiencies of up to 5.0:1. A possible explanation for the reduced ability of immobilised protein A to bind 5 times the stoichiometric amount of IgG are steric hindrance effects, for example due to poor accessibility of individual domains of the immobilised protein for the significantly larger antibody molecules. This may be due, for example, to the molecular topology and other properties of the support surface or alternatively to the nature and multiplicity of the chemical bonding of protein A to this surface. In addition, partial denaturing due to physical or chemical factors, in particular of the antibody-affine domains of protein A, is also a possible cause of the loss of function.

Since affinity media with immobilised protein A, in particular in their use for purification of diverse antibodies for pharmaceutical purposes by affinity chromatography, represent a considerable cost factor for the preparation processes carried out therewith owing to the high price of purified natural or recombinant protein A, there is considerable motivation to increase the economy of affinity media of this type by improving the efficiency of immobilised protein A. Such an improvement in the efficiency, for example to values greater than 1.5: 1, could result in higher throughput of the sample to be purified in the purification of IgG, based on the amount of immobilised protein A employed. In other words, a smaller amount of immobilised protein A would be required for the purification of a given amount of IgG for the same throughput per time unit, which would result in a reduction in costs for use of the process.

Accordingly, the object of the present invention is to immobilise protein A on suitable supports in such a way that an efficiency of greater than 1.5:1 is achieved with respect to utilisation of the affine binding sites present for IgG to protein A.

It has been found that affinity media with protein A chemically covalently immobilised on the surface can be produced with an efficiency for immobilised protein A of greater than 1.5:1, typically between 2:1 and 4:1. The improvement in the efficiency has been achieved in the immobilisation of protein A on hydroxyl-containing supports, where the binding of the protein to the surface of the support material has taken place via a particular spacer provided with suitable functional groups.

The present invention therefore relates to a hydrophilic support material to which protein A is covalently bonded via a spacer, characterised in that the spacer is derived from the group of aminocarboxylic acids of the formula I
H₂N—R—COOH

where

R═(CH₂)ₙ and

n=2-10,

where one or more H atoms in group R may be replaced, independently of one another, by linear or branched C₁-C₆-alkyl, F, Cl, OH, O—C₁-C₆-alkyl, SH, S—C₁-C₆-alkyl, NO₂, linear or branched C₁-C₆-alkyl-OH or NH—C₁-C₆-alkyl, in particular methyl, ethyl, propyl, methoxy, ethoxy, 2-hydroxyethyl or 2-hydroxypropyl, and

one or more non-adjacent methylene groups in group R may be replaced, independently of one another, by O, S, N-alkyl, NH, CONH or NHCO.

In a preferred embodiment, the support material carries hydroxyl groups at least on its surface.

In a preferred embodiment, protein A is bonded to the spacer via one of its primary amino groups by an amide bond.

In a preferred embodiment, R consists only of unsubstituted methylene groups.

In a particularly preferred embodiment, R═(CH₂)₄.

In a further preferred embodiment, the support material is porous having mean pore diameters of between 50 and 150 nm.

In a preferred embodiment, the support material is a hydrophilic polymer of 2,3-dihydroxypropyl allyl ether and methylenebisacrylamide as crosslinking agent and preferably has a porosity of approximately 80% and a mean pore diameter of between 80 and 100 nm.

In another preferred embodiment, the support material is porous silica gel.

The present invention also relates to a process for the preparation of support material with immobilised protein A, characterised by the following process steps:
a) provision of an activated hydrophilic support material
b) covalent bonding of a spacer of the formula I to the support material from step a)
   H₂N—R—COOH I
where
R═(CH₂)ₙ and
n=2-10,
where one or more H atoms in group R may be replaced, independently of one another, by linear or branched C₁-C₆-alkyl, F, Cl, OH, O—C₁-C₆-alkyl, SH, S—C₁-C₆-alkyl, NO₂, linear or branched C₁-C₆-alkyl-OH or NH—C₁-C₆-alkyl, in particular methyl, ethyl, propyl, methoxy, ethoxy, 2-hydroxyethyl or 2-hydroxypropyl, and
one or more non-adjacent methylene groups in group R may be replaced, independently of one another, by O, S, N-alkyl, NH, CONH or NHCO
c) covalent bonding of protein A to the spacers of the modified support material obtained from step b).

In a preferred embodiment, the bonding of the spacer to the support material takes place via its primary amino group by reaction with an epoxide group present on the support.

In another particularly preferred embodiment, the bonding of the spacer to the support material takes place as secondary amine via its primary amino group to an aldehyde group present on the support.

In a preferred embodiment, the bonding of protein A takes place to a carboxyl group of the spacer.

In a particularly preferred embodiment, the bonding of protein A takes place to a carboxyl group of the spacer which has previously been converted into an N-hydroxysuccinimide ester.

The present invention also relates to chromatography columns or extraction cartridges containing the support material according to the invention.

The present invention also relates to the use of the support material according to the invention for the analysis or isolation of antibodies.

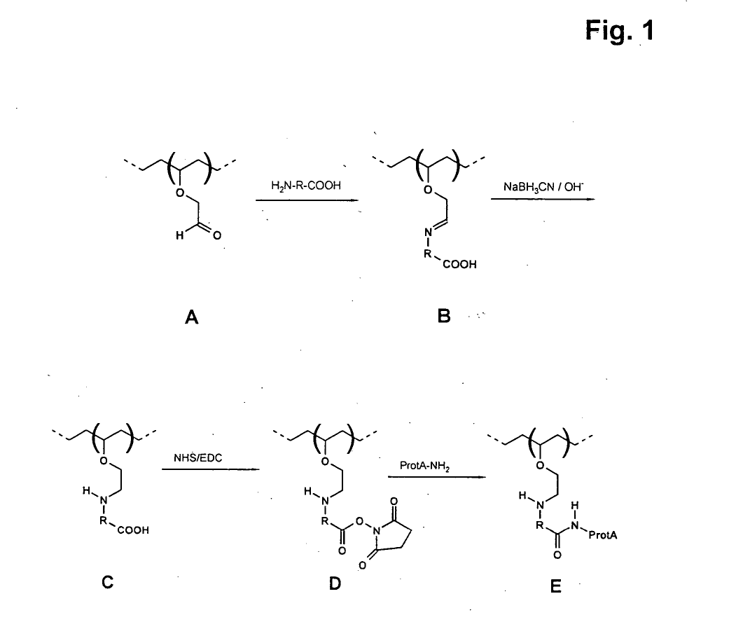

FIG. 1 shows by way of example a sequence of chemical reactions for the preparation of the support materials according to the invention with immobilised protein A.

Any type of antibody-binding protein A is suitable for the preparation of the support materials according to the invention, i.e., for example, the known natural form of antibody-binding protein A or genetically modified, so-called recombinant forms of this protein obtained from cell cultures with correspondingly transformed host cells.

Support materials which are suitable in accordance with the invention are solid or gelatinous materials, as typically employed for chromatographic or extraction purposes. These are typically organic or inorganic polymers which are hydrophilic, at least on their surface. The support materials preferably carry hydroxyl groups, at least on their surface. If the base polymer contains no hydroxyl groups, these can be introduced subsequently by corresponding modification or alternatively introduced as early as during synthesis of the polymer by copolymerisation with correspondingly functionalised monomers.

Examples of suitable support materials and/or base supports are homogeneously and/or heterogeneously crosslinked polystyrenes, homogeneously and/or heterogeneously crosslinked polyvinyl acetates, polyamides, polyethylenes, polypropylenes, crosslinked dextrans, crosslinked agarose, materials based on polysaccharides, such as cellulose or amylose, and support materials based on silica, titanium oxide, zirconium oxide and aluminum oxide.

A particularly preferred support material is a hydrophilic polymer of 2,3-di-hydroxypropyl allyl ether and methylenebisacrylamide as crosslinking agent. Particular preference is given to a polymer of this type having a porosity of approximately 80% and a mean pore diameter of between 80 and 100 nm. A material of this type is available from Merck KGaA under the name Fractoprep®.

It must be ensured that the support material selected cannot undergo any side reactions which adversely affect the binding of protein A. For example, a crosslinked polymethacrylate appears to react with NHS ester intermediates during immobilisation. In this case, other types of immobilisation must be selected or the support material must be correspondingly treated or modified.

Silica-based support materials are materials which consist entirely of glass, ceramics and/or silica (silica gel) or in which the surface is at least partially covered by glass, ceramics and/or silica. The term silica also encompasses materials prepared using silanes carrying one or two organic radicals (i.e., for example, C₁ to C₈-alkyl and/or C₅ to C₁₈-aryl radicals, in particular methyl, ethyl, n-isopropyl, n-tert-butyl, phenyl, benzyl or naphthyl), i.e. so-called hybrid materials.

The support material can be, for example, in the form of a monolithic column or capillary, plate, particle, coating, fibre, filter or another porous or nonporous structure. The material is preferably in the form of a porous structure, particularly preferably in the form of a particle or monolithic moulding. In the case of particulate materials, suitable materials are, for example, those having a particle size (mean diameter) of between 20 and 100 μm.

In the case of porous support materials, the pore structure is preferably monomodal or bimodal. Tri- or oligomodal pore distributions are also possible. Preference is given to mean pore diameters of between 30 and 300 nm, particularly preferably between 50 and 150 nm.

WO 95/03256 and particularly WO 98/29350 disclose processes for the production of inorganic monolithic mouldings by a sol-gel process. These materials contain mesopores having a diameter of between 2 and 100 nm and macropores having a mean diameter of greater than 0.1 μm and are thus highly suitable for use in accordance with the invention.

Processes for the introduction of hydroxyl groups on the surface of support materials are known to the person skilled in the art. In the case of silica-gel materials, use can be made, for example, of diol-modified phases.

For the purposes of the invention, the term “activated support materials” is taken to mean support materials containing reactive groups which are able to form a covalent bond with primary amines with or without addition of additional reagents. Corresponding activated support materials are also used, for example, for the introduction of separation effectors into support materials for chromatography. Examples of activated support materials are materials containing azlactone groups, NHS esters, epoxide groups or aldehyde groups. The person skilled in the art is aware of the reaction conditions and/or additional reagents that are necessary for forming a covalent bond between the spacer and a specific activated support.

For the purposes of the invention, a spacer is a molecule which can on the one hand be covalently bonded to the support material and to which, on the other hand, protein A can be covalently bonded. In this way, protein A is not bonded directly to the support material, but instead with a spatial separation caused by the spacer. The most important property of the spacer is thus that a functional group is present for bonding to the support material and a functional group is present for bonding of protein A. For bonding to the support material, a functional group is necessary which facilitates bonding to the support and which is stable during the further steps for immobilisation of protein A and under the subsequent chromatographic or extraction conditions. In accordance with the invention, the bonding takes place via a primary amino group, which reacts, for example, with an aldehyde function or an epoxide group on the support material. For bonding of protein A, the spacer carries a carboxyl function, which is able to react with primary amino groups of protein A.

The spacer according to the invention is selected from the group of aminocarboxylic acids of the formula I
H₂N—R—COOH I

where R═(CH₂)ₙ,

n=2-10,

where one or more H atoms in group R may be replaced, independently of one another, by linear or branched C₁-C₆-alkyl, F, Cl, OH, O—C₁-C₆-alkyl, SH, S—C₁-C₆-alkyl, NO₂, linear or branched C₁-C₆-alkyl-OH or NH—C₁-C₆-alkyl, in particular methyl, ethyl, propyl, methoxy, ethoxy, 2-hydroxyethyl or 2-hydroxypropyl, and

one or more non-adjacent methylene groups in group R may be replaced, independently of one another, by O, S, N-alkyl, NH, CONH or NHCO.

In a preferred embodiment, R consists only of unsubstituted methylene groups.

Examples of suitable spacers are 5-aminopentanoic acid (R═(CH₂)₄) or 6-aminohexanoic acid (R═(CH₂)₅). R is particularly preferably (CH₂)₃, i.e. the spacer is 4-aminobutyric acid (GABA).

The support materials according to the invention therefore have the following general structure of the formula II
Sup-HN—R—CO-ProtA II

where

Sup=support material

ProtA=protein A

R═(CH₂)ₙ and

n=2-10,

where one or more H atoms in group R may be replaced, independently of one another, by linear or branched C₁-C₆-alkyl, F, Cl, OH, O—C₁-C₆-alkyl, SH, S—C—C₆-alkyl, NO₂, linear or branched C₁-C₆-alkyl-OH or NH—C₁-C₆-alkyl, in particular methyl, ethyl, propyl, methoxy, ethoxy, 2-hydroxyethyl or 2-hydroxypropyl, and

one or more non-adjacent methylene groups in group R may be replaced, independently of one another, by O, S, N-alkyl, NH, CONH or NHCO.

It goes without saying that the support material does not have, as depicted diagrammatically in the formula II, only one spacer, but instead a multiplicity of spacers. Usually, a protein A is not bonded to all spacers. The chemical bond between the spacer and the support material or protein A is depicted by a broken line, since, depending on the chemical structure and/or activation of the support material or protein, the bonding can take place via different functionalities.

The present invention also relates to a process for the preparation of support material with immobilised protein A, characterised by the following process steps:
a) provision of an activated support material. To this end, the hydrophilic support materials which are suitable in accordance with the invention are usually firstly reacted with corresponding reagents for introduction of (amino-)reactive groups.
b) covalent bonding of a spacer according to the invention to the support material from step a)
c) covalent bonding of protein A to the spacers of the modified support material obtained from step b)

The covalent bonding of the spacer to the support material takes place by methods known to the person skilled in the art, depending on the type of functional groups on the support material and the type of functional groups of the spacer. A preferred possibility consists in using a support material containing diol groups present on the surface. All or some of these can then be converted into aldehyde groups, for example by oxidation using sodium metaperiodate. The primary amino group of the spacer is then bonded to this activated support material by reductive amination. For coupling of protein A, a terminal carboxyl group of the spacer is then employed. This is reacted with protein A using an N-hydroxysuccinimide ester prepared therefrom as an intermediate or another correspondingly activated form, with formation of an amide-like bond between the carboxyl group of the spacer and the primary amino groups of the protein surface. A support with covalently immobilised protein A is obtained. FIG. 1 shows the reaction scheme just described again in detail. The aldehyde-functionalised support material employed in step A is firstly prepared from diol groups of the surface of the support material by oxidation using sodium periodate.

A→B, formation of Schiff bases by coupling of aminocarboxylic acids to the aldehyde groups

B→C, reduction of the double bond of the Schiff bases to the secondary amine

C→D, activation of the terminal carboxyl group to the N-hydroxysuccinimide ester via an O-acylisourea derivative formed as an intermediate

D→E, formation of an amide bond between primary amino groups of protein A and the terminal carboxyl group, present in activated form as N-hydroxysuccinimide ester, of the spacer coupled to the support material as secondary amine.

Abbreviations: NHS=N-hydroxysuccinimide; EDC=N-(3-dimethylaminopropyl)-N′-ethylcarbodiimide

A further possibility for the preparation of the support materials according to the invention consists in the use of epoxy-activated support materials, which are reacted with amino groups of a spacer.

The support materials according to the invention with immobilised protein A exhibit an efficiency of >1.5. The efficiency is typically even >2; preferably >3. In this way, less protein A is required per unit of the antibody to be bound on use of the support materials according to the invention. Since protein A is very expensive, the reduction in the amount of protein A required plays a major role in economic terms.

The support material according to the invention can be employed for any form of affinity chromatography or extraction. It is typically employed in chromatography columns or extraction cartridges.

Even without further comments, it is assumed that a person skilled in the art will be able to utilise the above description in its broadest scope. The preferred embodiments and examples should therefore merely be regarded as descriptive disclosure which is absolutely not limiting in any way.

### EXAMPLES

1. Comparison of the Support Materials According to the Invention With the Prior Art

The following table shows the binding capacity, immobilised amount of protein A and efficiency thereof for various samples of the chromatography media according to the invention for affinity chromatography of immunoglobulins and of comparative samples. Preparation procedures for the materials mentioned are given in Examples 2 and 3.

TABLE 1

| | | | | Efficiency | |
|---|---|---|---|---|---|
| | | Capacity | Immobilised | protein A³ | |
| | | IgG¹ | protein A² | μmol/g of IgG/ | Base |
| No. | Spacer | mg/ml | mg/ml | μmol/g of iPA | material |
| S1 | — | 12.7 | 4.9 | 0.8 | FP |
| S2 | — | 12.5 | 3.8 | 1.2 | FP |
| S3 | — | 6.6 | 2.3 | 1.0 | FP |
| S4 | — | 14.6 | 6.2 | 0.7 | FP |
| S5 | — | 10.9 | 3.3 | 1.0 | FP |
| S6 | — | 18.1 | 5.5 | 1.0 | FP |
| S7 | — | 13.8 | 3.5 | 1.2 | FP |
| S8 | — | 13.7 | 4.7 | 0.9 | FP |
| S9 | — | 11.7 | 3.1 | 1.1 | FP |
| S10 | — | 17.2 | 4.6 | 1.1 | FP |
| S11 | — | 10.8 | 1.8 | 1.8 | FP |
| S14 | β-ALA | 7.1 | 0.8 | 2.6 | FP |
| S15 | GABA | 9.7 | 1.0 | 3.0 | FP |
| S16 | 5-AVA | 8.5 | 1.5 | 1.7 | FP |
| S17 | 6-AHA | 13.5 | 2.4 | 1.7 | FP |
| S18 | GABA | 5.2 | 0.4 | 4.2 | FP |
| S19 | GABA | 8.8 | 0.7 | 3.8 | FP |
| S20 | GABA | 10.1 | 1.0 | 2.9 | FP |
| S21 | GABA | 9.6 | 1.1 | 2.6 | FP |
| S24 | 6-AHA | 31.0 | 3.5 | 2.6 | Silica gel |
| | | | | | diol |

¹Binding capacities for human IgG (Gammanorm, Pharmacia, 10 mg/ml, 20 mM sodium phosphate buffer, pH 7.0), measured as bound and recovered protein, at 10% breakthrough; contact time for loading 4 minutes; column I.D. 1 cm, bed depth 18 mm)
²Determined by amino acid analysis after immobilised protein A has been cleaved off by hydrolysis for 12 hours with 6 M HCl.
³Calculated as mass ratio of bound IgG/immobilised protein A.

Samples S1-S11 are preparations in which protein A has been bonded directly to the surface of the Fractoprep support material by reductive amination. Various degrees of activation of the surface with aldehyde groups and different amounts of protein A immobilised thereto were used. Samples S14 to S17 are preparations prepared by the process according to the invention using different spacers (β-ALA=β-alanine, GABA=gamma-aminobutyric acid, 5-AVA=5-aminovaleric acid, 6-AHA=6-aminohexanoic acid). Samples S18 to S21 were prepared with GABA as spacer, but with increasing activity of the activated N-hydroxysuccinimide ester prepared as an intermediate for the coupling of protein A (see FIG. 1D). Sample S24 was prepared starting from a silica gel diol (Fuji Silysia 800 diol) with 6-AHA as spacer for immobilised protein A.

Table 2 below furthermore shows the corresponding data for commercially available protein A support materials:

TABLE 2

| | Capacity | Immobilised | | |
|---|---|---|---|---|
| | IgG¹ | protein A² | Efficiency | |
| Product | mg/ml | mg/ml | protein A³ | Manufacturer |
| Protein A | 21 | 7.9 | 0.8 | Amersham |
| Sepharose ® | | | | Biosciences |
| MAB Select ® | 26 | 5.3 | 1.5 | Amersham |
| | | | | Biosciences |
| Prosep ® rA | 30 | 7.7 | 1.2 | Millipore |
| POROS ® | 25.3 | 5.0 | 1.5 | Perseptive |
| 50 A | | | | |

The data given clearly show that the support materials according to the invention have significantly higher efficiencies than support materials according to the prior art.

2. Procedures for the Preparation of Protein A Supports in Accordance With the Prior Art Without Spacers

Procedure for the Preparation of Sample S1

The storage medium is removed from 20 ml of FRACTOPREP® raw material by washing ten times on a glass frit with 20 ml of water each time, and the filter cake is suspended in 40 ml of 0.8 M sodium metaperiodate solution and shaken at room temperature for 15 minutes. The gel is then filtered off from the supernatant liquid on a glass frit with suction and washed ten times with water and five times with 0.1 M sodium hydrogenphosphate buffer, pH 8.0, 0.15 M sodium chloride. The filter cake is suspended in a freshly prepared solution of 150 mg of protein A in 20 ml of 0.1 M sodium hydrogenphosphate, 0.15 M sodium chloride, pH 8.0.125 mg of sodium cyanoborohydride are then added, and the mixture is shaken at room temperature for 12 hours. The gel is again filtered off with suction and washed five times with 0.1 M sodium hydrogenphosphate, 0.15 M sodium chloride, pH 8.0, and then ten times with 20 mM sodium acetate buffer, pH 5.5. The gel is stored in 20 mM phosphate buffer, pH 7.0, with 0.01% of azide or alternatively in the same buffer containing 20% (v/v) of ethanol instead of azide, at 5°C.

Procedure for the Preparation of Sample S5

The sample is prepared analogously to sample S1, but with 75 mg instead of 150 mg of protein A.

Procedure for the Preparation of Sample S6

The sample is prepared analogously to sample S1, but with 0.2 M instead of 0.8 M sodium periodate solution.

Procedure for the Preparation of Sample S11

The sample is prepared analogously to sample S5, but with 0.022 M instead of 0.8 M sodium periodate solution.

3. Procedures for the Preparation of Support Materials According to the Invention

Procedure for the Preparation of Sample S15

The storage medium is removed from 20 ml of FRACTOPREP® raw material by washing ten times on a glass frit with 20 ml of water each time, and the filter cake is suspended in 40 ml of 0.2 M sodium metaperiodate solution and shaken at room temperature for 15 minutes. The gel is then filtered off from the supernatant liquid on a glass frit with suction and washed ten times with water and five times with 0.1 M sodium hydrogenphosphate buffer, pH 8.0, 0.15 M sodium chloride. The filter cake is suspended in a freshly prepared solution of 500 mg of 4-aminobutyric acid in 20 ml of 0.1 M sodium hydrogenphosphate, 0.15 M sodium chloride, pH 8.0. 125 mg of sodium cyanoborohydride are then added, and the suspension is shaken at room temperature for 16 hours. The gel is again filtered off with suction and washed five times with 0.1 M sodium hydrogenphosphate, 0.15 M sodium chloride, pH 8.0, and then ten times with 20 mM sodium acetate buffer, pH 5.5. 10 ml each of a freshly prepared solution of 1.4 g of EDC in 10 ml of 20 mM sodium acetate buffer, pH 5.5, and 10 ml of 0.2 g of N-hydroxysuccinimide in 10 ml of 20 mM sodium acetate buffer, pH 5.5, are added successively to the filter cake, which is suspended and shaken at room temperature for 1 hour. The solid is subsequently filtered off with suction, washed ten times with 20 ml of sodium acetate buffer (20 mM, pH 5.5) each time, and the subsequent step is carried out immediately. The filter cake is introduced into a solution of 150 mg of protein A in 25 ml of 20 mM sodium acetate buffer, pH 5.5, suspended therein and shaken at room temperature for 12 hours. The solid is subsequently filtered off with suction and washed 5 times with 20 mM sodium acetate buffer, pH 5.5, followed by five times with 20 mM phosphate buffer, pH 7.0. The product is stored in 20 mM phosphate buffer, pH 7.0, with 0.01% of azide or alternatively in the same buffer containing 20% (v/v) of ethanol instead of azide, at 5° C.

Samples S14 and 16-17 are prepared correspondingly, in each case using the spacer mentioned in Table 1.

Procedure for the Preparation of Sample S24

35 g of silica gel diol (Fuji Silysia MB 800-40/75) are suspended in 160 ml of a 0.2 M sodium metaperiodate solution and shaken at room temperature for 15 minutes. The gel is then filtered off with suction and washed ten times with water and five times with 0.1 M sodium hydrogenphosphate buffer, 0.15 M sodium chloride, pH 8.0. The aldehyde-activated silica gel obtained takes up a volume of about 80 ml. The washed gel sediment is suspended in a freshly prepared solution of 2.4 g of 6-aminohexanoic acid in 40 ml of 0.1 M sodium hydrogenphosphate, 0.15 M sodium chloride, pH 8.0. 500 mg of sodium cyanoborohydride, freshly dissolved in 40 ml of 0.1 M sodium hydrogenphosphate, 0.15 M sodium chloride, pH 8.0, are then added, and the suspension is shaken at room temperature for 16 hours. The solid is again filtered off with suction and washed five times with 0.1 M sodium hydrogenphosphate, 0.1 M sodium chloride, pH 8.0, and ten times with 20 mM sodium acetate buffer, pH 5.5. 40 ml each of a freshly prepared solution of 5.6 g of EDC in 10 ml of 20 mM sodium acetate buffer, pH 5.5, and 40 ml of 0.8 g of N-hydroxysuccinimide in 10 ml of 20 mM sodium acetate buffer, pH 5.5, are added successively to the filter cake, and the suspension is shaken at room temperature for one hour. The solid is subsequently filtered off with suction, washed ten times with 20 ml of 20 mM sodium acetate buffer, pH 5.5, each time and immediately thereafter the filter cake is introduced into a solution of 50 mg of protein A in 25 ml of 20 mM sodium acetate buffer, pH 5.5. The mixture is shaken at room temperature for 16 hours, and the solid is then filtered off with suction and washed five times with 20 mM sodium acetate buffer, pH 5.5, and five times with 20 mM sodium phosphate buffer, pH 7.0. The product is stored in 20 mM phosphate buffer, pH 7.0, with 0.01% of azide or alternatively in the same buffer containing 20% (v/v) of ethanol instead of azide, at 5°C.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever. In the foregoing and in the examples, all temperatures are set forth uncorrected in degrees Celsius and, all parts and percentages are by weight, unless otherwise indicated. The entire disclosures of all applications, patents and publications, cited herein and of corresponding German application No. 102004029341.4, filed Jun. 17, 2004 are incorporated by reference herein. The preceding examples can be repeated with similar success by substituting the generically or specifically described reactants and/or operating conditions of this invention for those used in the preceding examples. From the foregoing description, one skilled in the art can easily ascertain the essential characteristics of this invention and, without departing from the spirit and scope thereof, can make various changes and modifications of the invention to adapt it to various usages and conditions.

1. A hydrophilic support material with immobilized protein A comprising a protein A that is covalently bonded to a hydrophilic support material via a spacer, wherein the spacer is derived from the group of aminocarboxylic acids of formula I
H₂N—R—COOH I
where
R═(CH₂)ₙ and
n=2-10,
where one or more H atoms in R may be replaced, independently of one another, by linear or branched C₁-C₆-alkyl, F, Cl, OH, O—C₁-C₆-alkyl, SH, S—C₁-C₆-alkyl, NO₂, linear or branched C₁-C₆-alkyl-OH or NH—C₁-C₆-alkyl, and
one or more non-adjacent methylene groups in group R may be replaced, independently of one another, by O, S, N-alkyl, NH, CONH or NHCO.

2. A support material according to claim 1, wherein the support material carries hydroxyl groups at least on its surface.

3. A support material according to claim 1, wherein protein A is bonded to the spacer via a primary amino group of the protein A by an amide bond.

4. A support material according to claim 1, wherein R consists of unsubstituted methylene groups.

5. A support material according to claim 1, wherein R═(CH₂)₄.

6. A support material according to claim 1, wherein the support material is porous having mean pore diameters of 50 to 150 nm.

7. A support material according to claim 1, wherein the support material is a hydrophilic polymer of 2,3-dihydroxypropyl allyl ether and methylenebisacrylamide as crosslinking agent.

8. A support material according to claim 1, wherein the support material is porous silica gel.

9. A process for preparing a support material according to claim 1, comprising
a) providing an activated support material,
b) covalently bonding a spacer of formula I to the support material, and
c) covalently bonding protein A to the spacer.

10. A process according to claim 9, wherein the bonding of the spacer to the support material takes place via a primary amino group of the spacer by reaction with an epoxide group present on the support.

11. A process according to claim 9., wherein the bonding of the spacer to the support material takes place as secondary amine via a primary amino group of the spacer to an aldehyde group present on the support.

12. A process according to claim 9, wherein bonding of protein A takes place to a carboxyl group of the spacer.

13. A chromatography column or extraction cartridge containing a support material according to claim 1.

14. A method for analyzing or isolating antibodies comprising bringing together the antibodies with a support material according to claim 1.

15. A support material according to claim 1, wherein one or more H atoms in R are replaced by methyl, ethyl, propyl, methoxy, ethoxy, 2-hydroxyethyl or 2-hydroxypropyl.
